(19) United States
(12) Patent Application Publication
(10) Pub. No.: US 2014/0252926 A1
(43) Pub. Date: Sep. 11, 2014

(54) DIALYSIS MACHINE WITH SELF-SUPPORTING MACHINE HOUSING

(71) Applicant: B. Braun Avitum AG, Melsungen (DE)

(72) Inventors: OLIVER SCHAEFER, NEUENSTEIN (DE); KAI-UWE RITTER, REDNITZHEMBACH (DE)

(73) Assignee: B. Braun Avitum AG, Melsungen (DE)

(21) Appl. No.: 14/196,337

(22) Filed: Mar. 4, 2014

(30) Foreign Application Priority Data
Mar. 7, 2013 (DE) 10 2013 102 281.2

(51) Int. Cl. A61M 1/14

(52) U.S. Cl. CPC A61M 1/14
USPC 312/209

(57) ABSTRACT

The present invention relates to a medical device for extracorporeal blood treatment which is preferably configured as a mobile device. According to aspects of the present invention, the device includes a device housing which is configured as a self-supporting housing, preferably without any frame whatsoever.

### CROSS REFERENCE TO RELATED APPLICATIONS

This application claims priority to German Patent Application No. 10 2013 102 281.2 filed Mar. 7, 2013, the contents of such application being incorporated by reference herein.

### FIELD OF THE INVENTION

The present invention relates to a medical device for extracorporeal blood treatment, and in particular to a mobile dialysis machine with a frameless, self-supporting machine housing.

### BACKGROUND OF THE INVENTION

is Medical devices for extracorporeal blood treatment, in particular dialysis machines, are often configured as mobile units, which can be moved to spatially different sites located e.g. within a hospital or a dialysis center. Devices of this type are also regularly subjected to maintenance cycles, which are intended to guarantee perfect operational reliability, the maintenance work being not carried out on the ward, but in workshops which are specially equipped/established for this purpose and to which the devices have to be delivered.

To this end, dialysis machines are equipped with a carriage of their own, e.g. in the form of castors articulated on the side of the machine housing facing the floor. Alternatively, dialysis machines may, however, also be configured such that they do not include an (integrated) carriage of their own. In this case, the dialysis machine will have to be moved by means of an external, separate means of conveyance, e.g. by means of a dolly, etc.

Whenever dialysis machines are configured as mobile machines they are subjected to higher mechanical loads, which they must also resist for a prolonged period of time. In particular, the dialysis machines are moved back and forth by the staff maneuvering the machine housing thus causing shocks and vibrations which will also be transmitted to the housing and the device components accommodated therein. In addition, such dialysis machines must provide possibilities of accessing their interior machine components so that e.g. service or maintenance work can be carried out.

### DESCRIPTION OF THE RELATED ART

Medical devices for extracorporeal blood treatment, e.g. dialysis machines which are generally known from the prior art, comprise a plurality of different components from the fields of electronics, sensor technology, hydraulics and mechanics. There is a hydraulic pump-filtration system (dialysis fluid line system) including a plurality of hydraulic elements, such as pumps, valves, filters etc., pipings, containers and the like, as well as an electronic control comprising an adequate sensor system, said electronic control controlling the blood treatment process and adjusting the device such that it will work optimally. Some of these components are high-precision components and are therefore expensive to purchase and service. On the other hand, some of the components used are simple components, e.g. individual hoses, which are especially used for device sections provided as disposables.

Taking all this into account, the components installed are a substantial expense factor, which, in view of the sensitive technology and the necessary robustness of the device, cannot and must not be substantially reduced by using less expensive components. Another expense factor is to be seen in the assembly of medical devices of this type. As a matter of principle it can here be said that the smaller the number of assembly steps required is, the lower the manufacturing costs will be. Insofar, the overall cost for a dialysis machine according to the prior art is essentially determined by the two above mentioned pools of costs, so that an improvement concerning at least one of these pools of costs may have a substantial effect on the overall cost.

### SUMMARY OF THE INVENTION

In view of the above, it is an object of at least one aspect of the present application to provided a medical device for extracorporeal blood treatment, in particular a dialysis machine, which can be manufactured in less manufacturing steps and can thus be produced at a lower cost.

This object is achieved by a medical device for extracorporeal blood treatment, in particular a dialysis machine, having the features of claim 1. Advantageous embodiments of the invention are the subject matter of the subclaims.

The invention relates to the following inventive consideration:

In principle, it is known to provide medical devices for extracorporeal blood treatment with a machine housing comprising a supporting frame or lattice structure taking up the whole static (and also dynamic) loads acting on the device. The claddings of the housing, such as paneling/cladding boards and similar housing components, are normally only used as a decorative cover (dust protection/visual cover) or represent opening flaps or doors, which temporarily close e.g. inlets provided for the purpose of service and maintenance and which are preferably hinged to the frame structure.

Normally, all these components defining the housing are accommodated and held in position by the frame structure. Therefore, these components only have to support themselves and, possibly, comparatively small/light attachment parts, such as electronic circuit boards. The interior statics of the machine housing is, however, exclusively determined by the housing frame.

Such a housing frame normally consists of a plurality of carrier profiles having e.g. an L-, U- or T-shape, which are welded, screw-fastened and/or riveted to one another, said carrier profiles being coupled to one another after the principle of the triangle of forces and producing thus the housing stiffness that is necessary for absorbing external forces. Although such a machine housing thus has the robustness which, from the user's point of view, will be necessary for daily use, the production of such a machine housing is complicated and therefore also expensive. It will first be necessary to assemble/produce the housing frame, which is then clad/covered with the respective housing components.

The present invention has now discovered that a simplification of the machine housing is technically realizable without having to interfere with the functional design of the machine as regards the hydraulic and electronic components installed therein, and that the number of assembly steps can thus be reduced. As has already been explained hereinbefore, this has a considerable influence on the manufacturing costs, which represent one of the two major pools of costs.

The present invention is inspired by chassis construction in the field of vehicle manufacturing, in particular the manufacturing of automobiles and aircraft, where the principle of ladder frames has already been abandoned for some time and has been replaced by so-called self-supporting chassis. This technology shows that car bodies with high stiffness can be obtained by a body sheet arrangement which substantially follows the external and internal forces to be expected. This design principle is now transferred to the housing construction for the dialysis machine according to the present invention.

According to a first aspect, the present invention therefore discloses that a preferably mobile medical device for extracorporeal blood treatment should be provided with a substantially frameless device/machine housing of the self-supporting type. This housing preferably consists of a number of housing plates, which are welded, screw-fastened, riveted and/or glued to one another and which are connected to one another such that these housing plates will substantially only be acted upon by shear forces as a result of external forces acting thereon according to the normal device operation to be expected. The self-supporting housing has thus imparted thereto a stiffness comparable to that of a housing frame having the known structural design.

According to a further, possibly independent, optional aspect of the present invention, the fixedly interconnected housing plates are arranged such that apertures or openings are defined at predetermined locations of the housing, without (substantially) interrupting or impairing the progression or transmission of forces through the housing plates, i.e. the remaining fixedly interconnected housing plates are oriented and positioned relative to one another such that the basic principle of applying shear loads to the housing plates is substantially maintained and the overall stiffness of the housing will thus be weakened/impaired by the openings to the least possible extent.

According to a further, possibly independent, optional aspect of the present invention, the selected housing plates are partially reinforced so as to form e.g. articulation points for doors or flaps closing the openings or so as to stiffen areas which will (may) presumably have applied thereto forces that differ from the shear load direction of the housing plate in question. Preferably, at least selected reinforcements of this type are given a form/structure which makes them suitable for fulfilling additional functions, such as the function as a housing for accommodating fans or blowers, as a connection base for external/adaptive auxiliary devices and similar functional elements.

### BRIEF DESCRIPTION OF THE DRAWINGS

The invention is best understood from the following detailed description when read in connection with the accompanying drawings. Included in the drawings are the following figures:

FIG. 1 shows a schematic and functional diagram of one housing side of a medical device for extracorporeal blood treatment, in particular a dialysis machine according to a preferred embodiment of the present invention,

FIGS. 2 and 3 show in a perspective view the device housing according to a FIG. 1 with the front and rear doors open,

FIG. 4 shows in a perspective view the already mounted side parts and hood of the device housing according to FIG. 1,

FIG. 5 shows a lower section of the front of the device housing below the front door according to FIG. 2 or 3,

FIG. 6 shows the shear force progression to be expected in a side part of the device housing according to the present invention,

FIG. 7 shows the cross-section of a side part with integrated partial stiffenings (carrier profiles) for absorbing transverse/bending forces to be expected,

FIG. 8 shows the force progression to be expected in the hood of the housing and

FIG. 9 shows the force progression to be expected in the front and/or rear doors of the housing device according to the present invention in the case of a closed condition.

### DETAILED DESCRIPTION OF THE PREFERRED EMBODIMENTS

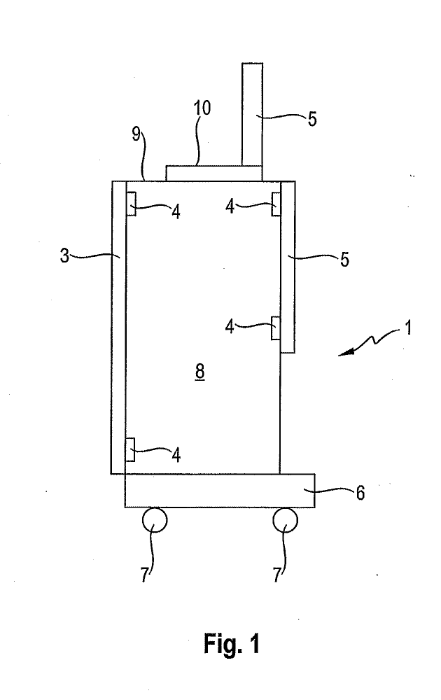

According to FIG. 1, the medical device for extracorporeal blood treatment, in particular the dialysis machine, according to the preferred embodiment of the present invention comprises a device or machine housing 1, which houses or is capable of housing a certain number of machine parts or machine components, such as a control electronics, at least parts of the hydraulic system, pumps, heating units and perhaps also reservoirs or bags for selected operating materials, which are all known from the prior art to a sufficient extent and which are therefore not shown in detail in the enclosed figures.

The machine housing 1 according to FIG. 1 comprises essentially two (one-piece) side plates 8, a rear wall 3 configured at least partially as a hinged door/flap, a hood 9 connecting the two (parallel spaced) side plates 8 at the top, a base plate (or base of the device) 6 connecting the two side plates 8 at the bottom, and a front wall configured at least partially as a hinged door/flap 2.

The hood 9 has centrally formed thereon or is provided with a connection base 10 on which an additional device, such as a monitor 5, can be mounted/is mounted. The base plate 6 has articulation points at three or four positions spaced in the circumferential direction of the housing. At these articulation points, castors 7 can be mounted/are mounted, said castors 7 defining, together with the base plate 6, a device-internal (integrated) carriage. The base plate 6 may, however, also be placed onto a separate carriage, e.g. a dolly.

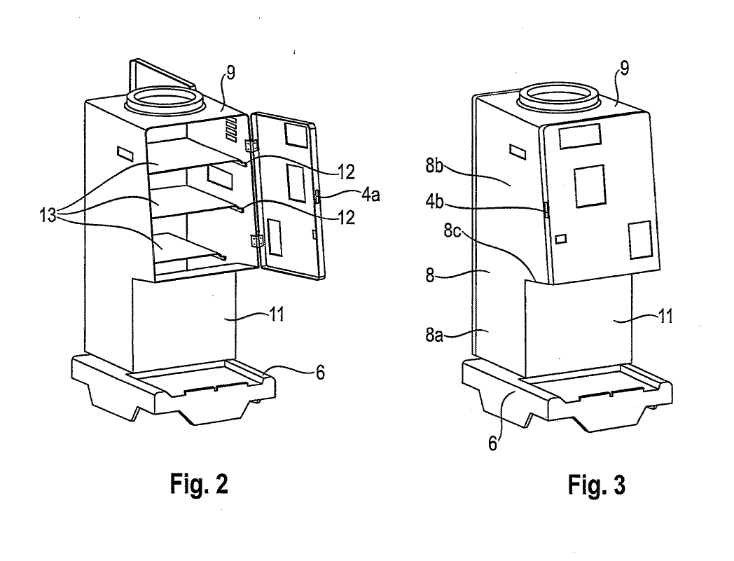

FIGS. 2 and 3 show a perspective view of the machine housing 1 as an engineering representation. FIG. 2 shows the housing doors/flaps 2, 3 at an open position and FIG. 3 shows them at a closed position.

Accordingly, the two parallel spaced side plates or side parts 8, which are preferably configured as one-piece components, can theoretically be subdivided into two vertically spaced plate sections 8a, 8b, viz. a lower section 8a of small depth and an upper section 8b of large depth, whereby a projection 8c is formed between the lower and the upper section. In the area of the lower section 8a the two side plates 8 are fixedly interconnected via a front plate 11, which is connected to the two side plates 8 by a substance-to-substance bond, by positive locking engagement or by a force fit connection. Preferably, the front plate 11 is welded to the two side plates 8 along the two vertical edges thereof. Basically, a connection between the front plate 11 and the two side plates 8 is, however, established such that the forces acting as transverse loads on the two side plates 8 can be introduced in the front plate 11 as shear forces, whereby the two side plates 8 will be stiffened in their transverse direction.

According to FIG. 1, the hood 9 and preferably also the base plate or the base 6 of the device are fixed to the upper and lower edges of the two side plates 8, preferably welded thereto along the edge length thereof, and also the front plate 11 has its lower edge fixedly connected to the base plate 6, and is preferably welded thereto along the edge length thereof. The two side plates 8, the hood 9, the base plate 6 and the front plate 11 thus provide a torsion-resistant housing, which is here fully open on the back and, at least in the area of the upper section 8b, open on the front.

As can be seen from FIG. 1 in combination with FIGS. 2 and 3, doors or flaps 2, 3 are arranged on the two above-mentioned openings, which are defined by assembling the individual plates (i.e. they are not cut out from the plates). These doors or flaps are here pivotably hinged on a respective side part (side plate) 8 and have closure mechanisms 4a at their vertical edges located opposite the hinges 4, said closure mechanisms 4a being adapted to be brought into engagement with complementary locks 4b provided on the respective other side part (side plate) 8. The doors/flaps 2, 3 are here (according to FIG. 1) attached to a respective first side part 8 and adapted to be locked to a respective second side part 8 such that, at the closed position of the respective door/flap 2, 3, the latter is adapted to take up shear loads and contributes thus to further stiffening of the housing 1, as can be seen from the force arrows shown in FIG. 9. Alternatively to the above-mentioned closure mechanisms, the doors/flaps 2, 3 may, of course, also be configured such that, when occupying their closed position, their longitudinal edge(s) are screw-fastened or clamped to the respective side part 8 and optionally the hood 9 and/or the base plate 6. In addition, the doors 2, 3 may be provided with a chamfer circumferentially extending on the edge side, whereby the warping resistance of the doors 2, 3 will be increased. This chamfer may encompass the two side plates 8, the hood 9 and the projection 8c thus causing a more stable coupling to these housing parts.

Finally, it can be seen from FIG. 2 that, at least in the upper sections 8b (optionally the also in the lower sections 8a), the two side plates 8 have attached thereto substantially horizontal rails used as support and/or slide rails for shelves or supporting plates 13. These horizontal rails may also be arranged such that they define (part of) a support to the base. Simultaneously, these support/slide rails act as stiffening means of the side plates 8 so as to avoid warping in response to an application of external forces.

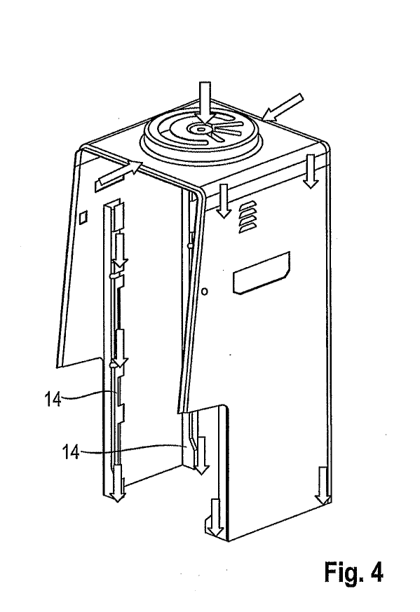

FIG. 4 shows the housing 1 according to the present invention in a partially assembled condition, said figures showing only the two side plates 8 and the hood 9 so that the inner sides of the side plates 8 can be seen more clearly.

As has already been indicated hereinbefore, the hood 9 comprises here the central connection base 10 in the form of an adapter to which e.g. the monitor 5 can be fitted. Simultaneously, the connection base 10 causes the hood 9 to be partially stiffened. In addition, the hood 9 is chamfered at two opposed edges in the present example, so that it can be brought into planar contact with the side plates 8. The hood 9 can thus be connected more easily to the side plates 8 and higher forces can be transmitted between the plates.

Finally, the two side plates 8 have on the inner surfaces thereof parallel vertical struts 14 (possibly alternatively or additionally to the horizontal rails 12), which are spaced apart in the direction of the depth of the housing and which are also provided for receiving/supporting thereon the shelves 13. To this end, the vertical struts 14 may be provided with horizontal slots. The vertical struts 14 may preferably consist of a hollow section (carrier section) within which cables and lines can be laid. Thus, this system also allows separate laying of the cables according to load and signal lines within the vertical struts. The cables laid therein can thus also be partially EMC shielded against the strut material.

In FIG. 4 the force progression in the side plates and the hood is indicated by force arrows. Accordingly, the vertical struts have the effect that the side plates are reinforced/stiffened for increased absorbance of vertical forces that may result e.g.

from the auxiliary device attached to the hood, so as to prevent warping of the side plates. The hood 9 is provided for absorbing horizontal forces (as shear forces), the connection base 10 increasing the capability of the hood 9 to absorb vertical forces (transverse forces).

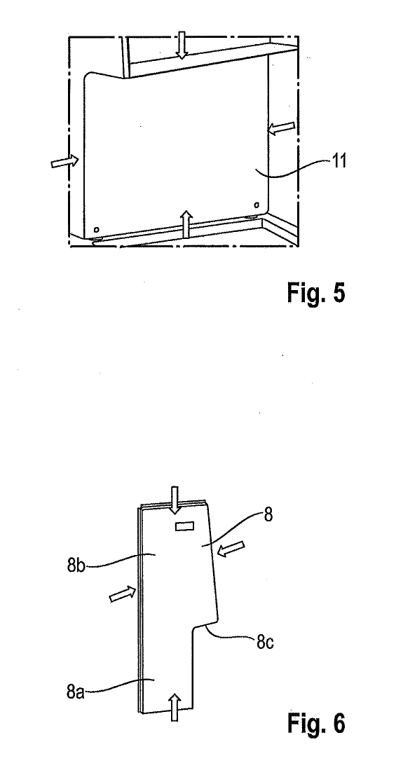

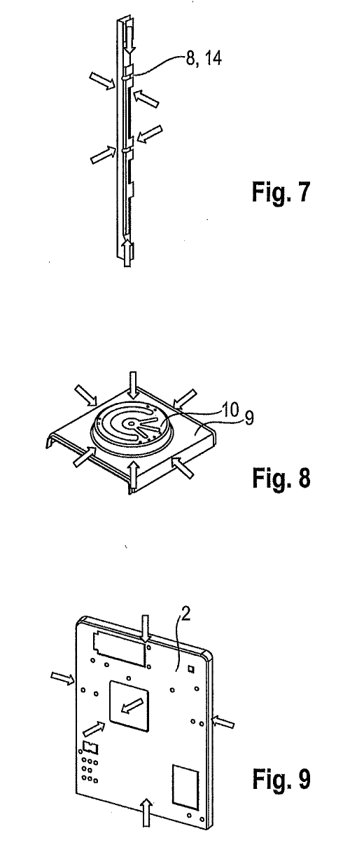

For making the loads on the housing 1, which will have to be expected as a result of normal handling of the dialysis machine, more easily understandable, the individual housing plates are shown separately and provided with respective force arrows in FIGS. 5 to 9.

Accordingly, the two side plates 8 will presumably have applied thereto substantially horizontal and vertical shear forces, which may partially also comprise transverse force components. These transverse force components are absorbed by the horizontal and/or vertical struts 12, 14 and/or distributed to both side plates 8 via the shelves.

The hood 9 has primarily applied thereto horizontal forces but, due to the integrated base 10, it is also capable of absorbing vertical forces and transmitting these forces to the side plates 8.

The front door 2 shown in FIG. 9 can only transmit forces in its closed condition. In this case it essentially absorbs vertical and horizontal forces as shear forces. The same applies to the front plate 11 below the front door 2 which, in the present embodiment according to FIG. 5, is not provided with any internal stiffening means and is therefore only adapted to have applied thereto horizontal and vertical forces as shear forces. In this context, it should, however, be mentioned that the front door 2 may also be provided with internal stiffening means and/or may have an adequate profile.

Summarizing, it can be said that the present invention relates to a medical device for extracorporeal blood treatment, which is preferably configured as a mobile device. According to the present invention, the device includes a device housing which is configured as a self-supporting housing, preferably without any frame whatsoever.

1. A medical device for extracorporeal blood treatment comprising a device housing in which at least hydraulic and electronic device components are housed or can be housed, wherein the device housing comprises a plurality of push cups, which are joined together such that a substantially frameless housing of the self-supporting type is formed.

2. The medical device for extracorporeal blood treatment according to claim 1, wherein the device housing includes a number of housing plates, which are fixedly interconnected to one another by a substance-to-substance bond, by positive locking engagement and/or by a force fit connection such that these housing plates will substantially only be acted upon by shear forces as a result of external forces acting thereon according to the normal device operation to be expected.

3. The medical device for extracorporeal blood treatment according to claim 2, wherein the housing plate connections are provided by welding, soldering, glueing, clinching, caulking, screw-fastening and/or riveting.

4. The medical device for extracorporeal blood treatment according to claim 2, wherein the fixedly interconnected housing plates are arranged such that at least one aperture or opening is defined at at least one predetermined location in the device housing.

5. The medical device for extracorporeal blood treatment according to claim 4, wherein the fixedly interconnected housing plates are arranged and oriented relative to one another such that, in spite of the at least one aperture or opening, the progression or transmission of forces through the housing plates will not be interrupted.

6. The medical device for extracorporeal blood treatment according to claim 4, wherein the fixedly interconnected housing plates are oriented and positioned relative to one another such that the basic principle of applying shear loads to the housing plates is substantially maintained.

7. The medical device for extracorporeal blood treatment according to claim 2, wherein selected housing plates are partially reinforced so as to form articulation points for doors and/or flaps and/or so as to stiffen areas which will presumably have applied thereto forces that differ from the shear load direction of the housing plate in question.

8. The medical device for extracorporeal blood treatment according to claim 7, wherein at least selected reinforcements have a form/structure which makes them suitable for fulfilling additional functions, wherein the additional functions include a housing for accommodating fans or blowers, as a connection base for adaptive auxiliary devices, and/or supporting plates and similar functional elements.

9. The medical device for extracorporeal blood treatment according to claim 7, wherein the hinged doors and/or flaps are adapted to be locked to the housing such that they will be incorporated in the distribution of forces in the housing and will thus serve to additionally stiffen the housing in the closed condition.

10. The medical device for extracorporeal blood treatment according to claim 1, wherein the device is a mobile dialysis machine with an integrated carriage.

11. The medical device for extracorporeal blood treatment according to claim 10, characterized in that the carriage comprises a plurality of castors, among which selected castors are supported on the housing such that they are vertically rotatable, the castors being placed on a base plate of the housing.
